(19) United States
(12) Patent Application Publication
(10) Pub. No.: US 2019/0246888 A1
(43) Pub. Date: Aug. 15, 2019

(54) ILLUMINATING DEVICE

(71) Applicant: OLYMPUS CORPORATION, Tokyo (JP)

(72) Inventors: Kazuaki TAMURA, Hachioji-shi (JP); Satoshi OHARA, Hachioji-shi (JP)

(73) Assignee: OLYMPUS CORPORATION, Tokyo (JP)

(21) Appl. No.: 16/393,127

(22) Filed: Apr. 24, 2019

Related U.S. Application Data
(63) Continuation of application No. PCT/JP2016/082827, filed on Nov. 4, 2016.

(51) Int. Cl. A61B 1/06; H01S 5/40; A61B 1/07

(52) U.S. Cl. CPC A61B 1/0653; H01S 5/4093; A61B 1/063; A61B 1/07; H01S 5/4012

(57) ABSTRACT

An illuminating device includes a light source unit configured to emit primary light, and an illuminating unit configured to generate illumination light based on the primary light. The illuminating unit includes a light converter configured to convert optical characteristics of the primary light. The light converter includes a light transmitter through which the primary light is transmitted, and a light diffuser formed within the light transmitter. The light diffuser is configured to diffuse part of the primary light, generating secondary light included in the illumination light. The light diffuser has a refractive index different from a refractive index of the light transmitter. The light diffuser has a hole formed within the light transmitter, a refractive index-modifying portion, or a crack portion.

### CROSS-REFERENCE TO RELATED APPLICATIONS

This application is a Continuation Application of PCT Application No. PCT/JP2016/082827, filed Nov. 4, 2016, the entire contents of which are incorporated herein by reference.

### BACKGROUND OF THE INVENTION

### 1. Field of the Invention

The present invention relates generally to an illuminating device including an illuminating unit.

### 2. Description of the Related Art

For example, Jpn. Pat. Appln. KOKAI Publication No. 2011-248022 discloses an illuminating system including a single-line optical fiber. This illuminating system includes an oval diffuser disposed on the distal end face of the optical fiber, in order to apply laser light (primary light guided by the optical fiber) as illumination light over a wide range. The diffuser is a separate member from the optical fiber.

### BRIEF SUMMARY OF THE INVENTION

An illuminating device includes a light source unit configured to emit primary light, and an illuminating unit configured to generate illumination light based on the primary light, emitting the illumination light to an opposite side of the light source unit. The illuminating unit includes a first light converter configured to convert optical characteristics of at least part of the primary light, and a holder internally holding the first light converter. The holder has a holder entrance portion where the primary light enters, and a holder exit portion where the illumination light exits. The first light converter includes a light transmitter through which the primary light is transmitted, and at least one light diffuser formed within the light transmitter. The light diffuser is configured to diffuse the at least part of the primary light traveling within the light transmitter, generating secondary light included in the illumination light. The light diffuser has a refractive index different from a refractive index of the light transmitter. The light diffuser has any one of a hole formed within the light transmitter, a refractive index-modifying portion, and a crack portion.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view of an illuminating device including an illuminating unit according to a first embodiment.

FIG. 2A is a view schematically showing the illuminating unit according to the first embodiment.

FIG. 2B is a view schematically showing the progressions of primary light, secondary light, and illumination light in the illuminating unit.

FIG. 3A is a view showing an example of a light diffuser of the illuminating unit.

FIG. 3B is a view showing an example of a light diffuser.

FIG. 3C is a view showing an example of a light diffuser.

FIG. 4A is a schematic view of an endoscope system on which the illuminating device is to be mounted.

FIG. 4B is a view showing a configuration of the endoscope system shown in FIG. 4A.

FIG. 5A is a view schematically showing Modification 1 of the illuminating unit.

FIG. 5B is a view schematically showing Modification 2 of the illuminating unit.

FIG. 5C is a view schematically showing Modification 3 of the illuminating unit.

FIG. 5D is a view schematically showing Modification 4 of the illuminating unit.

FIG. 5E is a view schematically showing Modification 5 of the illuminating unit.

FIG. 5F is a view schematically showing Modification 6 of the illuminating unit.

FIG. 6A is a schematic view of an illuminating device including an illuminating unit according to a second embodiment.

FIG. 6B is a view schematically showing the illuminating unit according to the second embodiment.

FIG. 6C is a view schematically showing a modification of the illuminating unit according to the second embodiment.

FIG. 7A is a view schematically showing an illuminating unit according to a third embodiment.

FIG. 7B is a view schematically showing a modification of the illuminating unit according to the third embodiment.

### DETAILED DESCRIPTION OF THE INVENTION

Hereinafter, embodiments of the present invention will be described with reference to the drawings. In some drawings, illustrations of parts of members are omitted for illustrative clarification purposes.

A central axis of primary light PL that enters a light transmitter 71 from a holder entrance portion 83a is referred to as a “central axis C”. A central axis C direction indicates, for example, a direction from the holder entrance portion 83a toward a holder exit portion 83b.

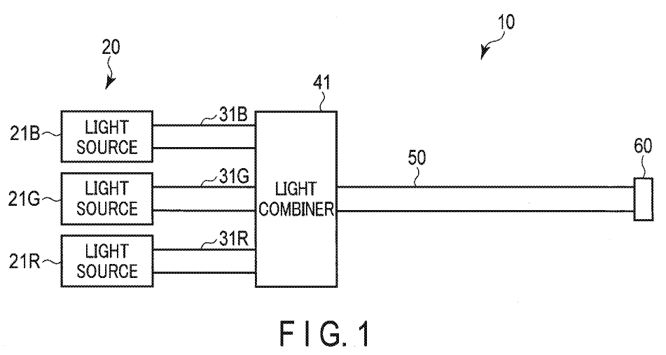

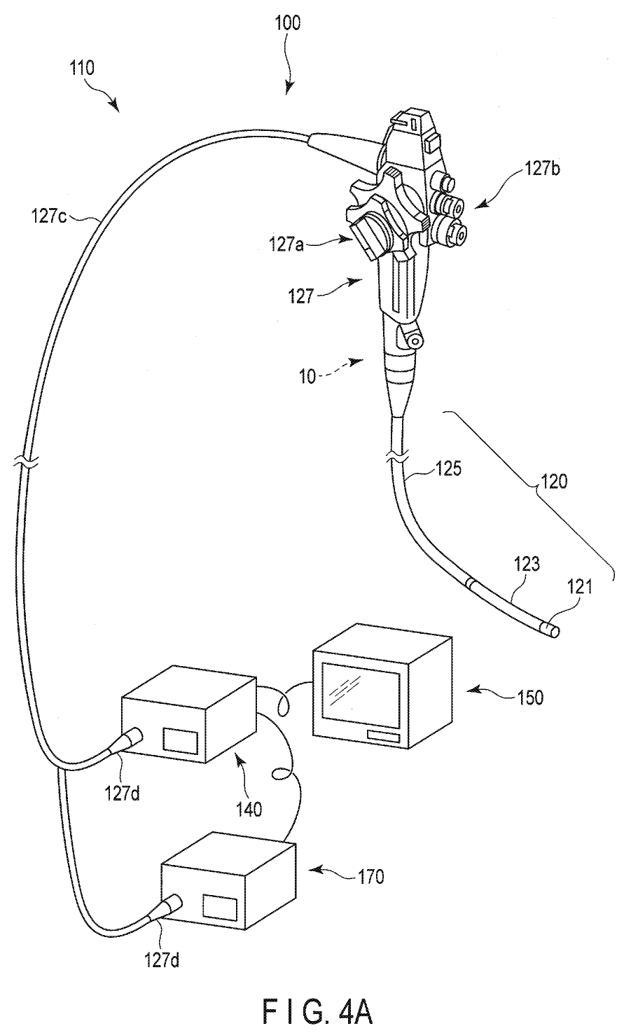

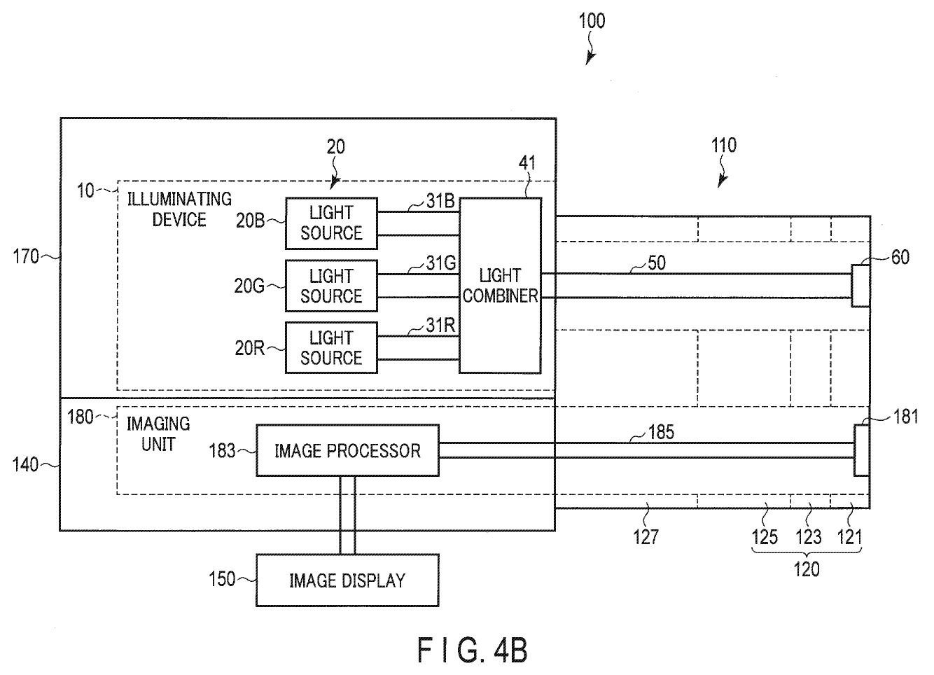

An illuminating device 10 shown in FIG. 1 will be described as an example, which is an endoscope illuminating device to be mounted, for example, on an endoscope system 100 shown in FIG. 4A. The illuminating device 10 may be mounted, for example, on a microscope or may function as a single device.

Illumination light IL indicates light emitted from an illuminating unit 60 to the outside of the illuminating unit 60. The illumination light IL includes light other than primary light PL (e.g., secondary light SL or tertiary light TL).

### First Embodiment

[Configuration]

Hereinafter, a first embodiment of the present invention will be described.

As shown in FIG. 1, an illuminating device 10 includes a light source unit 20, a light guide 50, and an illuminating unit 60.

The light source unit 20 emits beams of laser light having wavelengths different from each other, as primary light PL. The light source unit 20 includes, for example, light sources 21B, 21G, and 21R; light guides 31B, 31G, and 31R; and a light combiner 41.

The light source 21B includes, for example, a laser diode configured to emit a beam of blue laser light. The central wavelength of the beam of blue laser light is, for example, 445 nm.

The light source 21G includes, for example, a laser diode configured to emit a beam of green laser light. The central wavelength of the beam of green laser light is, for example, 532 nm.

The light source 21R includes, for example, a laser diode configured to emit a beam of red laser light. The central wavelength of the beam of red laser light is, for example, 635 nm.

The light guide 31B is optically coupled to the light source 21B and the light combiner 41, and guides a beam of laser light emitted from the light source 21B to the light combiner 41.

The light guide 31G is optically coupled to the light source 21G and the light combiner 41, and guides a beam of laser light emitted from the light source 21G to the light combiner 41.

The light guide 31R is optically coupled to the light source 21R and the light combiner 41, and guides a beam of laser light emitted from the light source 21R to the light combiner 41.

The light guides 31B, 31G, and 31R include, for example, multi-mode single-line optical fibers.

A condenser lens (not shown) is disposed between the light source 21B and the light guide 31B. Light emitted from the light source 21B is converged on the light guide 31B by the condenser lens. For this point, the same applies to a combination of the light source 21G and the light guide 31G, and a combination of the light source 21R and the light guide 31R.

The light combiner 41 combines beams of laser light having wavelengths different from each other and guided by the light guides 31B, 31G, and 31R. If the wavelengths of the beams of laser light are different from each other and the beams of laser light have blue, green, and red wavelengths as described above, then the combined light will become, for example, white light. The light combiner 41 is optically coupled to the light guide 50, and emits the combined white light as primary light PL toward the light guide 50. With this configuration, a white light observation can be performed by the single-line light guide 50.

The light combiner 41 includes, for example, an optical fiber combiner. With this configuration, the light combiner 41 can efficiently and compactly combine the beams of laser light. The light combiner 41 may include a spatial optical system including a lens and a dichroic mirror, for example.

Light sources to be used are not limited to the light sources 21B, 21G, and 21R. When four or more light sources are arranged, this enables a white light observation using white light with high color-rendering properties. When a light source configured to emit a beam of blue-violet laser light and the light source 21G are used, this enables a special light observation with the use of light absorption properties of hemoglobin. In the special light observation, blood vessels are highlighted and displayed. When a light source configured to emit near infrared light is used, this enables an observation using near infrared light. A light source to be used may be selected according to the observation.

The light source unit 20 may emit a beam of laser light having a wavelength as primary light PL, or may emit beams of laser light each having a wavelength as primary light PL. In this case, each of the light sources 21B, 21G, and 21R includes a laser diode configured to emit a beam of laser light of the same wavelength.

The light guide 50 guides the primary light PL emitted from the light source unit 20 to the illuminating unit 60. Therefore, the light guide 50 is optically coupled to the light combiner 41 and the illuminating unit 60. The light guide 50 guides the primary light PL from the light combiner 41 to the illuminating unit 60.

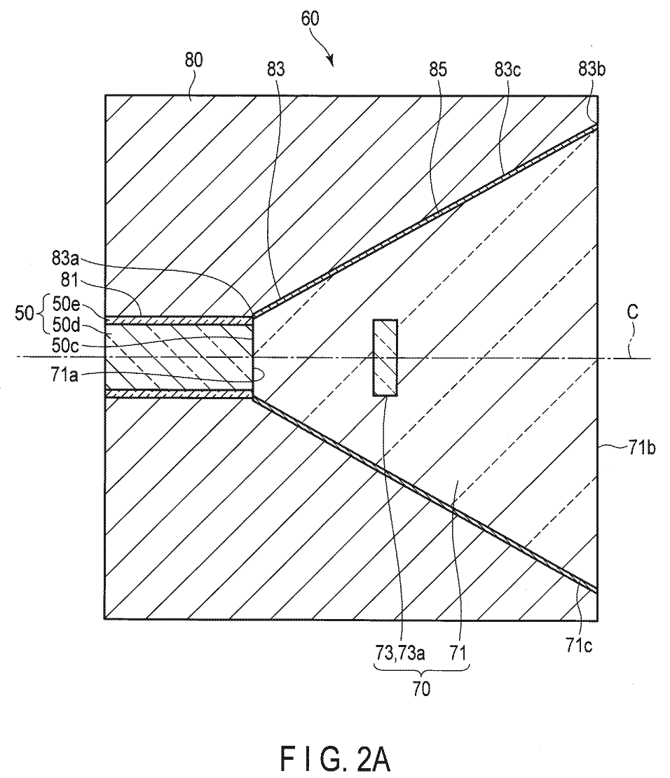

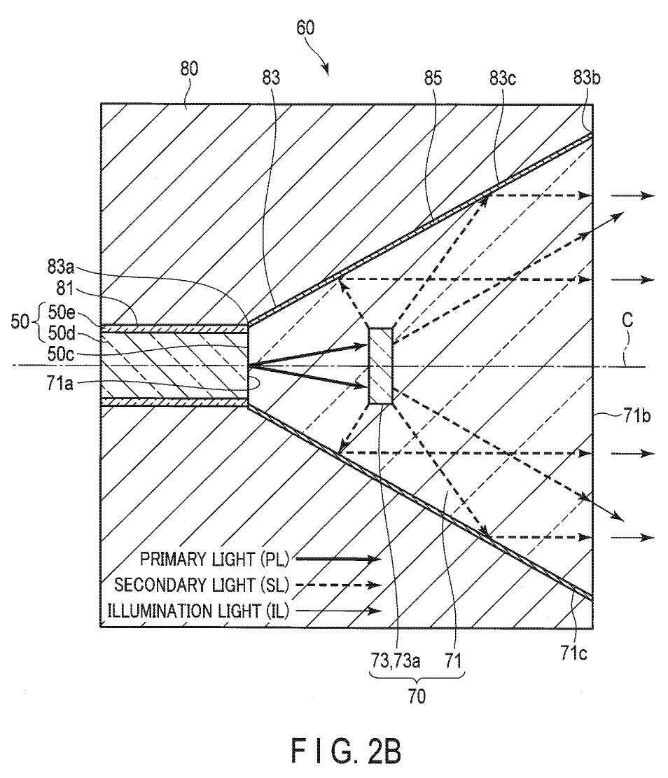

The outer diameter of the light guide 50 ranges from, for example, tens of micrometers (μm) to several hundred micrometers (μm). The light guide 50 is, for example, an optical fiber of a multimode fiber. For example, the core diameter of the optical fiber is 50 μm, and the numerical aperture (NA) is 0.2. Since laser light is used as primary light PL, a single-line optical fiber is used for the light guide 50. However, a bundle fiber may be used for the light guide 50. The material of the light guide 50 is, for example, a quartz glass, plastic, or resin. The light guide 50 is a rod-like member. The light guide 50 is an elongated member bendable by an external force. The light guide 50 has an entrance end that is optically coupled to the light combiner 41 and where the primary light PL emitted from the light combiner 41 enters and an exit end disposed on an opposite side of the entrance end. As shown in FIG. 2A, the exit end has an exit end face 50c, which is orthogonal to the central axis of the light guide 50. The exit end face 50c is a plane from which the primary light PL is emitted to the illuminating unit 60. The side face of the light guide 50 is parallel to the central axis of the light guide 50.

As shown in FIG. 2A, the light guide 50 has a core 50d configured to guide primary light PL and a clad 50e disposed on the outer circumference of the core 50d and having a refractive index lower than that of the core 50d. Due to the difference in refractive index between the core 50d and the clad 50e, the clad 50e has a function of trapping the primary light PL in the core 50d. For example, the distal end face of the core 50d and the distal end face of the clad 50e are placed on the same plane, which is orthogonal to the central axis of the core 50d. The distal end face of the core 50d and the distal end face of the clad 50e are included in the exit end face 50c. The light guide 50 may have a jacket (not shown), which is provided on the outer circumference of the clad 50e. The jacket improves the mechanical strength of the light guide 50, such as, tensile resistance and bending resistance. For the jacket, for example, a resin, such as nylon, acrylic, polyimide, and ETFE, is used.

As shown in FIG. 1, the illuminating unit 60 is disposed at the exit end of the light guide 50, which is disposed on the opposite side of the light source unit 20. The illuminating unit 60 is optically coupled to the light source unit 20 through the light guide 50, the light guides 31B, 31G, and 31R; and the light combiner 41. The illuminating unit 60 receives primary light PL emitted from the light source unit 20. The illuminating unit 60 generates illumination light IL based on the received primary light PL to emit the illumination light to the opposite side of the light source unit 20. For example, the illuminating unit 60 emits the illumination light IL to the outside of the illuminating unit 60. The illuminating unit 60 emits the illumination light IL in front of the illuminating unit 60 in the outside of the illuminating unit 60. Specifically, the illuminating unit 60 emits the illumination light IL from the holder exit portion 83b in front of the holder exit portion 83b. The front indicates, for example, the right side of the plane of sheet in FIGS. 1, 2, and 3, and indicates the opposite side of the position where the light source unit 20 and the light guide 50 are arranged in the central axis C direction. The illumination light IL of the present embodiment includes primary light PL and secondary light SL, or is secondary light.

As shown in FIG. 2A, the illuminating unit 60 includes a first light converter 70 configured to convert optical characteristics of at least part of the received primary light PL, generating secondary light SL, and a holder 80 internally holding the exit end of the light guide 50 and the first light converter 70. The exit end and the first light converter 70 are provided within the holder 80. The exit end, the first light converter 70, and the holder 80 are arranged rotationally symmetrically about the central axis C.

The first light converter 70 includes a light transmitter 71 through which the primary light PL and the secondary light SL are transmitted, and at least one light diffuser 73 that is formed within the light transmitter 71 in order to generate secondary light SL to be included in illumination light IL. In the present embodiment, it is assumed that one light diffuser 73 is disposed. The light diffuser 73 generates secondary light SL based on primary light PL, and details thereof will be described later.

The light transmitter 71 has, for example, a truncated conical shape. The light transmitter 71 has a small circular entrance face 71a that is optically coupled to the exit end face 50c of the light guide 50 and where the primary light PL emitted from the exit end face 50c enters, a large circular exit face 71b facing the entrance face 71a, and a side face 71c that is an outer circumferential face between the entrance face 71a and the exit face 71b. The entrance face 71a is of the same size as the exit end face 50c or larger than the exit end face 50c. The exit face 71b is exposed to the outside and emits illumination light IL. The entirety of the side face 71c contacts with the inner circumferential face 83c of the holder 80.

For example, the light transmitter 71 includes a transparent member having high transmittance with respect to the primary light PL and the secondary light SL. The light transmitter 71 needs to have a characteristic of transmitting the primary light PL and the secondary light SL.

The holder 80 has, for example, a cylindrical shape. The holder 80 is made of, for example, metallic brass. The holder 80 may include a metal such as aluminum or copper, or a metal compound such as aluminum nitride. The holder 80 includes a hollow portion 81 in which the exit end of the light guide 50 is placed, and a hollow portion 83 in which the light transmitter 71 is disposed. The hollow portions 81 and 83 are continuous within the holder 80 with respect to each other in the central axis C direction. The hollow portions 81 and 83 are through-holes extending through the within of the holder 80 in the central axis C direction. For example, the hollow portion 81 has a cylindrical shape, and the hollow portion 83 has a truncated conical shape.

The diameter of the hollow portion 81 is slightly larger than the diameter of the exit end of the light guide 50. The exit end is fixed to the hollow portion 81 by adhesion.

The hollow portion 83 communicates with the holder entrance portion 83a and the holder exit portion 83b. The holder entrance portion 83a is an opening portion where the light transmitter 71 on the entrance face 71a side is disposed and the primary light PL enters. The holder exit portion 83b is an opening portion where the light transmitter 71 on the exit phase 71b side is disposed and the illumination light IL exits. The diameter of the hollow portion 83 gradually expands from the holder entrance portion 83a toward the holder exit portion 83b in the central axis C direction. In other words, the hollow portion 83 has a truncated conical shape that increases in diameter from the holder entrance portion 83a to the holder exit portion 83b. An inner circumferential face 83c of the holder 80 in the hollow portion 83 is a tapered face.

The holder 80 includes a reflecting member 85, which is disposed on the inner circumferential face 83c of the holder 80 and configured to reflect the primary light PL and secondary light SL toward the holder exit portion 83b. The reflecting member 85 preferably has a high reflectance with respect to the primary light PL and the secondary light SL. When the primary light PL and the secondary light SL strike the reflecting member 85, the reflecting member 85 regularly reflects or diffusely reflects the primary light PL and the secondary light SL. The reflecting member 85 is fixed to the side face 71c of the light transmitter 71 by an adhesive, such as resin having high transmittance.

The reflecting member 85 may be disposed on at least part of the inner circumferential face 83c. For example, in the inner circumferential face 83c where the reflecting member 85 is not disposed, the side face 71c of the light transmitter 71 is fixed to the inner circumferential face 83c using an adhesive.

The reflecting member 85 in the present embodiment is, for example, a metal-reflecting film (a reflecting mirror) formed on the inner circumferential face 83c by thinly plating a metal, such as silver or aluminum. The reflecting member 85 may be protected by a protective film (not shown). The protective film covers the reflecting member 85. The protective film is a member having a high transmittance, such as a metal oxide film like a silicon dioxide or conductive glass.

Here, a general diffusion phenomenon in a light diffuser 73 will be described. The diffusion phenomenon is roughly categorized into Mie scattering and Rayleigh scattering.

Mie scattering occurs when the diameter of the light diffuser 73 is substantially the same as the wavelength of the primary light PL. The Mie scattering causes a large amount of a forward-scattering component, indicating a component that the secondary light SL scatters (travels) in front of the light diffuser 73, and a small amount of a backward-scattering component, indicating a component that the secondary light SL scatters (travels) to the rear of the light diffuser 73.

Rayleigh scattering occurs when the diameter of the light diffuser 73 is approximately 1/10 or less of the wavelength of the primary light PL. In Rayleigh scattering, the forward-scattering component is approximately identical to the backward-scattering component.

Considering the brightness of the illumination light IL emitted forward from the holder exit portion 83b, it is preferable in the present embodiment to use Mie scattering in which the amount of the forward-scattering component is larger than the amount of the backscatter component. On the other hand, when multicolor primary light PL is caused to scatter, it is necessary to consider the wavelength dependency of scattering. It is generally considered that the wavelength dependency of Mie scattering is higher than that of Rayleigh scattering. In order to eliminate color unevenness of the illumination light IL, Rayleigh scattering is preferable.

Regardless of whether Mie scattering or Rayleigh scattering is used, not only the forward-scattering component but also the backscattering component is generated when the secondary light SL is itself generated. That is, the secondary light SL travels around the light diffuser 73. The forward-scattering component is used as the illumination light IL, but the backward-scattering component does not contribute to the illumination light IL.

Then, as shown in FIG. 2B, for example, the reflecting member 85 reflects the secondary light SL that has radiated the reflecting member 85 to the holder exit portion 83b so that the secondary light SL travels to the holder exit portion 83b without entering the light diffuser 73 again by reflection.

For example, it is assumed that the secondary light SL has traveled from the light diffuser 73 to the reflecting member 85 disposed between the light diffuser 73 and the holder exit portion 83b in the central axis C direction, and is then reflected by the reflecting member 85. The secondary light SL is included in the forward-scattering component. At this time, the reflecting member 85 reflects the secondary light SL traveling from the light diffuser 73 to the holder exit portion 83b side to the holder exit portion 83b so that the secondary light SL travels to the holder exit portion 83b without entering the light diffuser 73 again.

For example, it is assumed that the secondary light SL has traveled from the light diffuser 73 to the reflecting member 85 disposed between the light diffuser 73 and the holder entrance portion 83a in the central axis C direction, and is then reflected by the reflecting member 85. The secondary light SL is included in the backward-scattering component. At this time, the reflecting member 85 reflects the secondary light SL traveling from the light diffuser 73 to the holder entrance portion 83a side to the holder exit portion 83b so that the secondary light SL travels to the holder exit portion 83b without entering the light diffuser 73 again. In this way, the reflecting member 85 reflects part of the secondary light SL so that the part of the secondary light SL included in the backward-scattering component, traveling from the light diffuser 73 toward the holder entrance portion 83a side, travels to the holder exit portion 83b without entering the light diffuser 73 again. In other words, the reflecting member 85 reflects, to the holder exit portion 83b, the secondary light SL traveling from the light diffuser 73 to the holder entrance portion 83a side so that the secondary light SL that has traveled from the light diffuser 73 to the holder entrance portion 83a side and then reflected by the reflecting member 85 travels to the holder exit portion 83b without entering the light diffuser 73 again.

As shown in FIG. 2B, in order to generate secondary light SL to be included in the illumination light IL, the light diffuser 73 diffuses, as secondary light SL, at least part of the primary light PL traveling within the light transmitter 71 and radiating the light diffuser 73. When the primary light PL having a very narrow light distribution is applied to the light diffuser 73, the primary light PL is diffused by the light diffuser 73, so that diffused light that is secondary light SL having a wide light-distribution angle is generated by the diffusion. In other words, the secondary light SL is diffused primary light PL (diffused light) that is diffused by the light diffuser 73. In the present embodiment, the diffusion includes refraction. In order to diffuse the primary light PL, a difference in refractive index is required at a boundary face between the light diffuser 73 and the light transmitter 71, which is a close contact member in close contact with the light diffuser 73. Therefore, the light diffuser 73 has a refractive index different from a refractive index of the light transmitter 71. The light diffuser 73 converts the primary light PL into secondary light SL having a different light-distribution angle from that of the primary light PL without converting the wavelength of the received primary light PL. The light diffuser 73 generates secondary light SL having a light-distribution angle equal to or larger than an NA of a core 50d of the light guide 50 according to the diffusion conditions of the light diffuser 73. The diffusion conditions of the light diffuser 73 include, for example, a difference in between a refractive index of the light diffuser 73 and a refractive index of the light transmitter 71, and the size of the light diffuser 73.

The light diffuser 73 is formed within the light transmitter 71, for example, by laser processing. Therefore, the light transmitter 71 includes an inorganic material such as a glass or ceramic having a Mohs hardness of 2 or more, or a resin such as acrylic having a Rockwell hardness of M50 or more. The light diffuser 73 may be formed by laser processing before the light transmitter 71 is disposed in the hollow portion 83, or by laser processing after the light transmitter 71 is disposed in the hollow portion 83. The laser light for use in the laser processing may be applied from either the entrance face 71a, the exit face 71b, or the side face 71c of the light transmitter 71. The laser light for use in the laser processing may be applied from the outer circumferential surface of the holder 80 holding the light transmitter 71. The laser light for use in the laser processing is different from the laser light emitted from the light source unit 20.

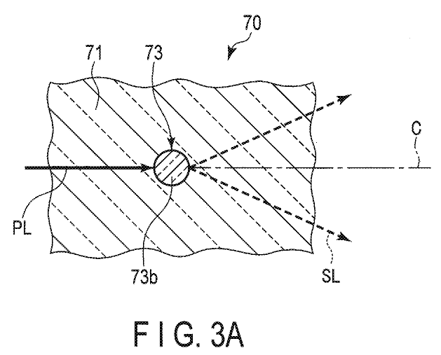

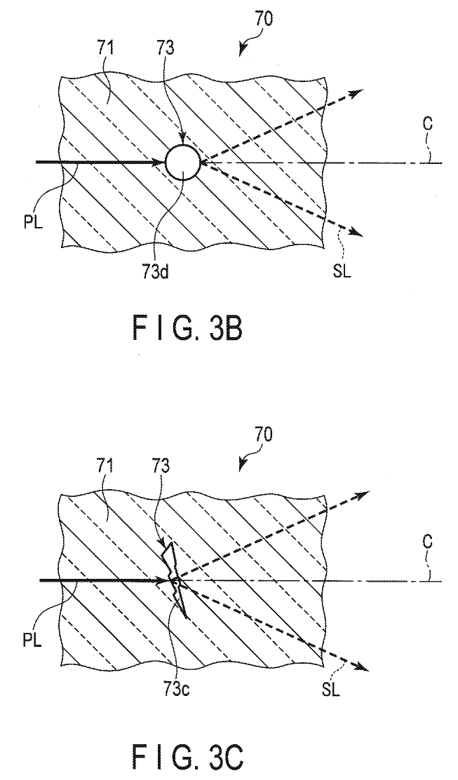

For example, the light diffuser 73 has a substantially columnar shape (see FIGS. 2A and 2B). The light diffuser 73 may also have a substantially spherical shape (see FIGS. 3A and 3B).

As shown in FIG. 2A, at least part of the light diffuser 73 is placed on the central axis C of the primary light PL that enters the light transmitter 71 from the holder entrance portion 83a. In the present embodiment, the central axis of the light diffuser 73 is placed on the central axis C. For example, the diameter of the light diffuser 73 is the same as the diameter of the core 50d of the light guide 50. The diameter of the light diffuser 73 may be smaller than the diameter of the core 50d. On the central axis C, the distance between the light diffuser 73 and the holder entrance portion 83a is shorter than the distance between the light diffuser 73 and the holder exit portion 83b. That is, the light diffuser 73 is disposed between the holder exit portion 83b and the holder entrance portion 83a to be nearer the holder entrance portion 83a than the holder exit portion 83b so that the primary light PL emitted in a state of having a light-distribution angle defined by an NA peculiar to the optical fiber is applied to the light diffuser 73. That is, the disposed position of the light diffuser 73 is determined based on the light-distribution angle of the primary light and the size of the light diffuser 73.

When the diameter of the light diffuser 73 is the same as the diameter of the core 50d, most of the primary light PL radiates the light diffuser 73. When the diameter of the light diffuser 73 is smaller than the diameter of the core 50d, part of the primary light PL radiates the light diffuser 73, and the remaining part of the primary light PL travels through the light transmitter 71 without entering the light diffuser 73. As described above, the light diffuser 73 is irradiated with at least part of the primary light PL traveling within the light transmitter 71.

The light diffuser 73 has various parts or various shapes for diffusion. As shown in FIG. 2A, the light diffuser 73 may have a hole 73a. As shown in FIG. 3A, the light diffuser 73 may have a refractive index-modifying portion 73b having a refractive index higher than that of the light transmitter 71, which is a close contact member. The light diffuser 73 may have a crack portion 73c (see FIG. 3C). Such a light diffuser 73 is formed, for example, by laser processing.

For example, when laser processing is performed only to part of the within of the light transmitter 71, this part evaporates, so that a hole 73a (see FIG. 2A), such as a pore, is formed. The hole 73a is a space filled with gas or a vacuum space. The shape and size of the hole 73a are adjusted according to the condensed spot diameter, the energy density, and the irradiation time of laser light to be used for the laser processing.

For example, when laser processing is performed only to part of the within of the light transmitter 71, the part is modified, and the refractive index of the part is increased as compared with the refractive index of the other part of the light transmission part 71 in which the laser processing is not performed. The refractive index-modified portion 73b shown in FIG. 3A is part of the light transmitter 71 modified by laser processing. The shape of the refractive index-modified portion 73b is not particularly limited. The refractive index is adjusted according to the condensed spot diameter, the energy density, and the irradiation time of laser light used for the laser processing.

For example, when laser processing is performed only to part of the within of the light transmitter 71, the shape of this part changes to a substantially columnar shape (see FIGS. 2A and 2B) or a substantially spherical shape (see FIG. 3B) by laser processing. The portion whose shape has changed functions as a light diffuser 73 having a substantially columnar shape or a substantially spherical shape. As shown in FIG. 3B, the light diffuser 73 in this case is a space 73d that is formed within the light transmitter 71 and covered with the light transmitter 71. The shape and size of the space 73d are adjusted according to the condensed spot diameter, the energy density, and the irradiation time of laser light used for laser processing. When the light diffuser 73 has a substantially columnar shape (for example, a substantially cylindrical shape), the central axis of the light diffuser 73 needs not be orthogonal to the central axis C.

For example, when laser processing is performed only to part of the light diffuser 73, a clack is generated in this part by laser processing. The portion where the clack is generated functions as a crack portion 73c (see FIG. 3C). The size and shape of the crack portion 73c are adjusted according to the condensed spot diameter, the energy density, and the irradiation time of laser light used for laser processing.

The refractive index of each of the hole portion 73a, the light diffuser 73 having a substantially columnar shape, the light diffuser 73 having a substantially spherical shape, the crack portion 73c, and the space 73d is adjusted according to the condensed spot diameter, the energy density, and the irradiation time of laser light used for laser processing. By such laser processing, the light diffuser 73 is formed in a state where it has a refractive index different from the refractive index of the light transmitter 71, and the size of the light diffuser 73 has been adjusted.

For example, with the light diffuser 73 having a substantially columnar shape, the light diffuser 73 is disposed on a plane orthogonal to the central axis C. The length of the light diffuser 73 on the central axis C is shorter than the length of the light diffuser 73 in a direction orthogonal to the central axis C.

The light diffuser 73 is formed within the light transmitter 71, but is not limited thereto. The light diffuser 73 may extends through the light transmitter 71. The position and the direction of extension of the light diffuser 73 are not particularly limited, as long as the primary light PL entered from the entrance face 71a can be directly applied to the light diffuser 73.

With reference to FIGS. 4A and 4B, an endoscope system 100 on which the illuminating device 10 is mounted will be described.

The endoscope system 100 includes, for example, an endoscope 110 configured to illuminate an observation object portion with illumination light IL, imaging the observation object portion, and a control device 140 configured to be detachably connected to the endoscope 110. The observation object portion is, for example, an affected part or a lesion in a body cavity. The endoscope system 100 includes an image display 150, which is a monitor configured to be connected to the control device 140 and display an observation object portion imaged by the endoscope 110, for example, and a light source device 170 configured to be detachably connected to the endoscope 110 and detachably connected to the control device 140. The endoscopic system 100 includes an imaging unit 180 configured to image an observation object portion.

The endoscope 110 includes, for example, an elongated hollow insertion section 120 to be inserted into a body cavity, and a grip section 127 coupled to the proximal end section of the insertion section 120 and configured to be gripped by an operator.

The insertion section 120 has a distal end hard section 121, a bendable section 123, and a flexible tube section 125 from the distal end side of the insertion section 120 toward the proximal end side of the insertion section 120. The proximal end of the distal end hard section 121 is coupled to the distal end of the bendable section 123, and the proximal end of the bendable section 123 is coupled to the distal end of the flexible tube section 125.

The flexible tube section 125 extends from the grip section 127. The grip section 127 has a bend control section 127a configured to operate the bendable section 123, a switch portion 127b for air supply/water supply, suction, and imaging, and a universal cord 127c configured to be connected to the grip section 127.

The universal cord 127c extends from a side face of the grip section 127. An end portion of the universal cord 127c is branched, and each of the end portions is provided with a connector 127d. One connector 127d is attachable to and detachable from the control device 140, and the other connector 127d is attachable to and detachable from the light source device 170.

The control device 140 controls the illuminating device 10, the endoscope 110, the image display 150, the light source device 170, and the imaging unit 180.

The imaging unit 180 includes an imager 181 configured to image an observation object portion, an imaging cable 185 configured to transmit an image imaged by the imager 181, and an image processor 183 configured to process the image transmitted by the imaging cable 185. The image processed by the image processor 183 is displayed by the image display 150. The imager 181 is disposed in the distal end hard section 121, the image processor 183 is disposed in the control device 140, and the imaging cable 185 is disposed in the endoscope 110. The imager 181 includes, for example, a CCD or CMOS. The image processor 183 is constituted by, for example, a hardware circuit including an ASIC, etc. The image processor 183 may be constituted by a processor. When the image processor 183 is constituted by a processor, an internal memory (not shown) or an external memory, to which the processor is accessible, is disposed in the control device 140. The internal memory or external memory stores program codes for causing the processor to function as the image processor 183 when executed by the processor.

The light source unit 20 is mounted on the light source device 170. The light guide 50 and the illuminating unit 60 are incorporated in the endoscope 110. Specifically, the exit end of the light guide 50 and the illuminating unit 60 are arranged in the distal end hard section 121.

[Operation]

The beams of laser light emitted from the light sources 21B, 21G, and 21R are guided to the light combiner 41 by the light guides 31B, 31G, and 31R. The beams of laser light are combined by the light combiner 41. The primary light PL, which is combined light, is guided to the illuminating unit 60 by the light guide 50.

As shown in FIG. 2B, the primary light PL is emitted from the exit end face 50c toward the first light converter 70. The light distribution of the primary light PL emitted from the exit end face 50c is narrow, and for example, the light-distribution half-value angle thereof is approximately 15 degrees. The intensity of the primary light PL on the central axis C is the highest, and the primary light PL lowers in intensity with distance from the central axis C in a direction orthogonal to the central axis C.

The primary light PL enters the light transmitter 71 from the holder entrance portion 83a (entrance face 71a) and travels within the light transmitter 71. Then, the primary light PL travels toward the light diffuser 73.

At least part of the primary light PL that has entered the light diffuser 73 is converted, by the light diffuser 73, into secondary light SL traveling in directions different from the direction of the beam of the primary light PL (e.g., in directions away from the central axis C) without the wavelength of the primary light PL being changed. With this, the light-distribution angle of the at least part of the primary light PL that has entered the light diffuser 73 is widened. That is, the primary light PL is converted into secondary light SL having a light-distribution angle equal to or larger than the NA of the core 50d of the light guide 50. Part of the primary light PL may be transmitted through the light transmitter 71, transmitted through the light diffuser 73, and then travel towards the holder exit portion 83b (exit face 71b) without being diffused by the light diffuser 73, which is not illustrated, though.

As shown in FIG. 2B, the light diffuser 73 is formed within the light transmitter 71. Therefore, the secondary light SL travels within the light transmitter 71. At least part of the secondary light SL is transmitted through the light transmitter 71 and then travels from the light diffuser 73 directly toward the holder exit portion 83b (exit face 71b).

Part of the secondary light SL included in the forward-scattering component travels toward the front of the light diffuser 73 and toward the holder exit portion 83b (exit face 71b) side of the reflecting member 85. The holder exit portion 83b (exit face 71b) side of the reflecting member 85 indicates a portion of the reflecting member 85 disposed between the light diffuser 73 and the holder exit portion 83b in the central axis C direction. After the secondary light SL is reflected by this portion of the reflecting member 85, the secondary light SL travels toward the holder exit portion 83b (exit face 71b) without entering the light diffuser 73 again by the reflection.

Other part of the secondary light SL included in the backward-scattering component travels toward the rear of the light diffuser 73 and toward the holder entrance portion 83a (entrance face 71a) side of the reflecting member 85. The rear indicates the light source unit 20 side in the central axis C direction. The holder entrance portion 83a (entrance face 71a) side of the reflecting member 85 indicates a portion of the reflecting member 85 disposed between the light diffuser 73 and the holder entrance portion 83a in the central axis C direction. After the secondary light SL is reflected by this portion of the reflecting member 85, the secondary light SL travels toward the holder exit portion 83b (exit face 71b) without entering the light diffuser 73 again by the reflection.

The reflection by the reflecting member 85 is performed at least once.

The primary light PL and the secondary light SL that have reached the holder exit portion 83b (exit face 71b) are emitted as illumination light IL from the holder exit portion 83b (exit face 71b) toward the outside. The illumination light IL is emitted forward from the holder exit portion 83b (the exit face 71b).

The inner circumferential face 83c of the holder 80 on which the reflecting member 85 is disposed is expanded in diameter from the holder entrance portion 83a to the holder exit portion 83b. Therefore, when the secondary light SL travels toward the rear of the light diffuser 73 and travels toward the holder entrance portion 83a (entrance face 71a) side of the reflecting member 85 and then is reflected by the reflecting member 85, the beam of the secondary light SL becomes to have a narrow radiation angle.

A portion of the reflecting member 85 that is disposed between the light diffuser 73 and the holder exit portion 83b in the central axis C direction reflects the secondary light SL to the holder exit portion 83b so that the secondary light SL travels to the holder exit portion 83b without entering the light diffuser 73 again. Therefore, the illumination light IL is efficiently extracted.

[Effect]

In the present embodiment, a diffuser that is separate from the light guide and that is configured to generate secondary light SL is not directly disposed on the distal end face of the light guide. In the present embodiment, the light diffuser 73 is formed within the light transmitter 71, and the first light converter 70 including the light transmitter 71 is disposed within the holder 80 in a state of being disposed on the exit end face 50c of the light guide 50, and the first light converter 70 is held by the holder 80. In other words, the light diffuser 73 is surrounded by the light transmitter 71 and the holder 80, is protected by the light transmitter 71 and the holder 80, and is not exposed to the outside. Therefore, it is possible to prevent the light diffuser 73 from being damaged by an externally applied physical load, such as an external force, to prevent deformation of the light diffuser 73, and to reduce fluctuations in light-distribution characteristics.

Furthermore, even if one of multiple various kinds of loads, such as a thermal load, a physical load, or a chemical load, is externally applied to the illuminating unit 60, the light diffuser 73 is protected by the light transmitter 71 and the holder 80. Therefore, it is possible to prevent the light diffuser 73 from being defective due to the load and to reduce fluctuations in light-distribution characteristics. Therefore, it is possible to reliably diffuse primary light PL, which is laser light, to prevent the laser light from being directly emitted to the outside, and to use secondary light SL, which is diffused light, as illumination light IL having a wide light distribution. As described above, in the present embodiment, it is possible to provide an illuminating device 10 with fewer fluctuations in light-distribution characteristics with respect to externally-applied physical loads including an external force, thereby allowing an illuminating device 10 with higher reliability to be provided.

At least part of the light diffuser 73 is placed on the central axis C. Therefore, it is possible to reliably convert the optical characteristics of at least part of the primary light PL.

The intensity of the primary light PL on the central axis C is highest, and the primary light PL lowers in intensity with distance from the central axis C in a direction orthogonal to the central axis C. In the present embodiment, at least part of the light diffuser 73 is placed on the central axis C. Therefore, a portion with the highest density in the primary light PL can be reliably diffused by the light diffuser 73, and the primary light PL can be efficiently converted into the secondary light SL.

The diffusion in the light diffuser 73 can reduce speckle, which is a phenomenon peculiar to laser light.

The secondary light SL having a light-distribution angle equal to or larger than the NA of the core 50d of the light guide 50 can be generated by the light diffuser 73.

By virtue of the reflecting member 85, it is possible to adjust the light-distribution characteristics of the secondary light SL, and to provide illumination light IL having desired light-distribution characteristics.

Modifications 1 to 6 of the illuminating unit 60 of the present embodiment will be described below. The drawings used for Modifications 1 to 6 illustrate examples of primary light PL, secondary light SL, and illumination light IL in the light traveling through the illuminating unit 60.

[Modification 1]

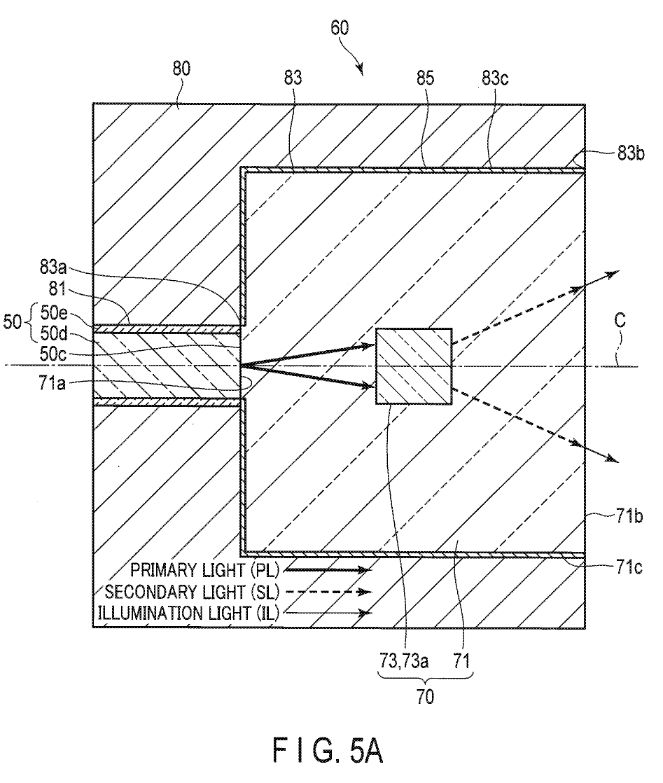

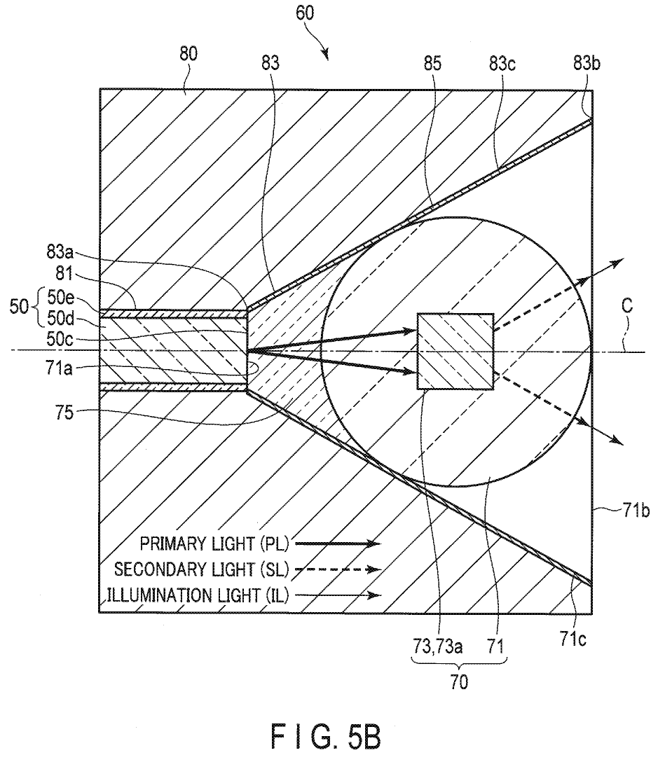

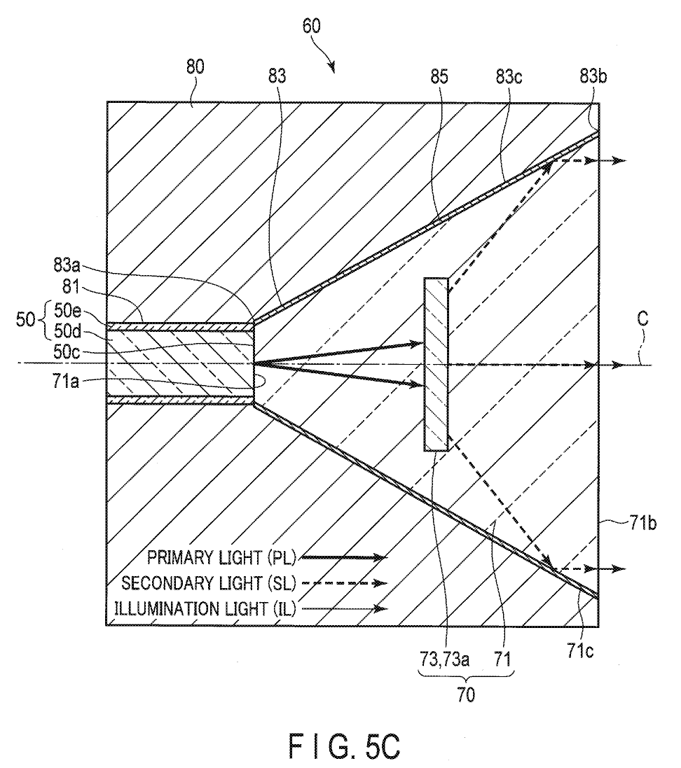

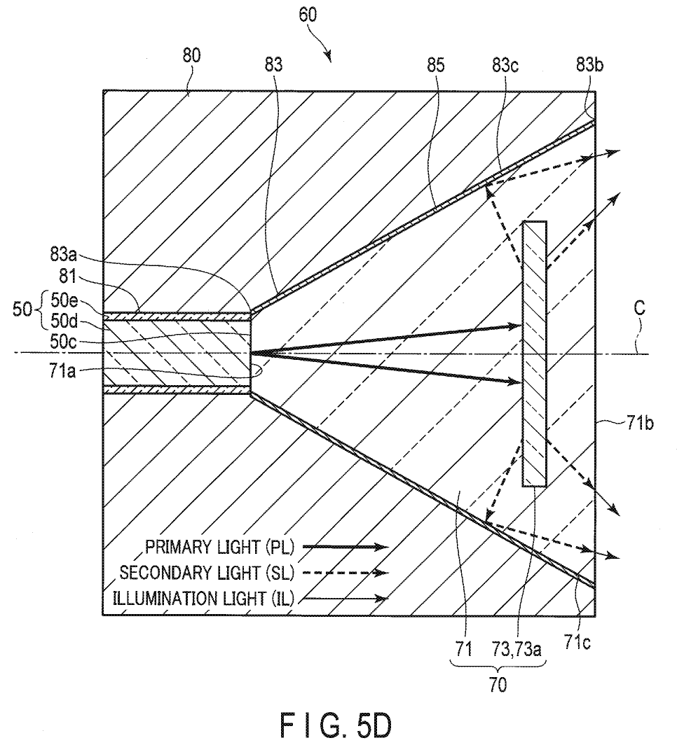

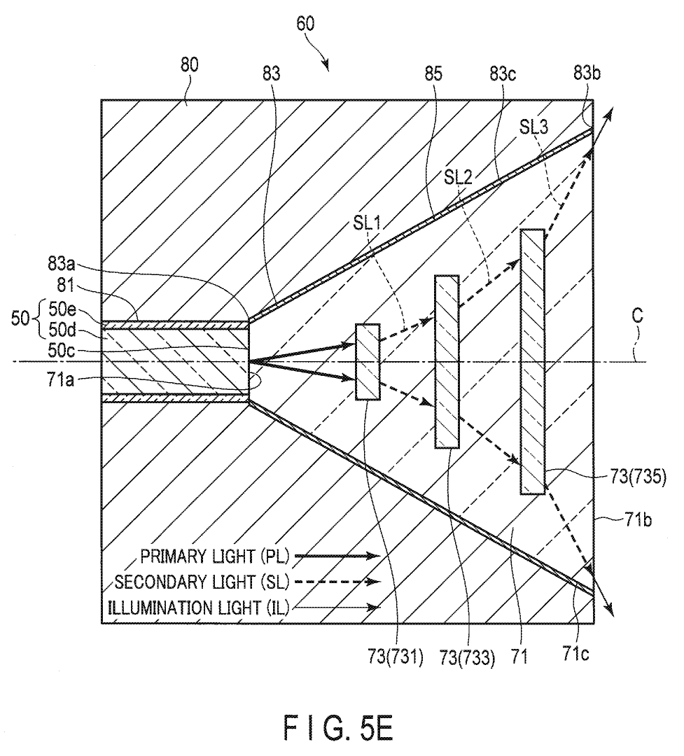

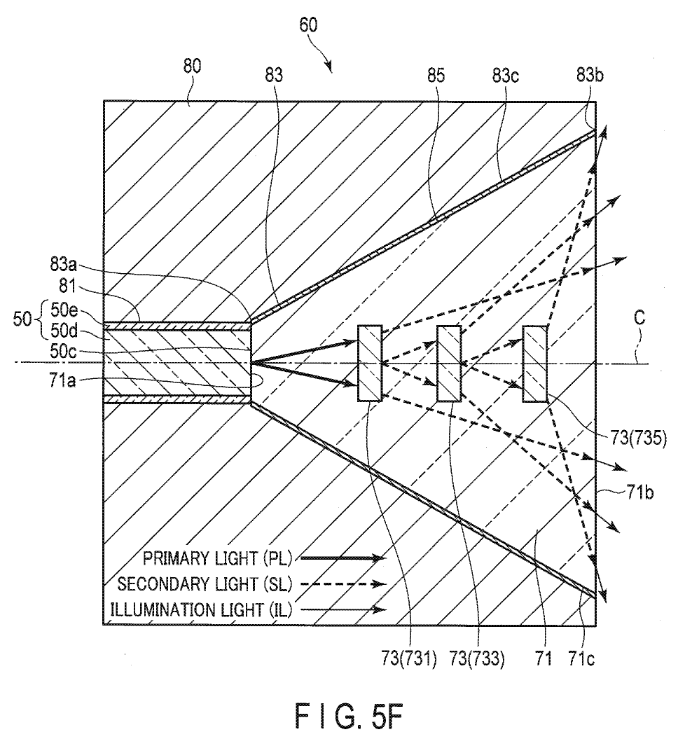

As shown in FIG. 5A, a hollow portion 83 has a substantially columnar shape. A light transmitter 71 has a substantially columnar shape and engages with the hollow portion 83. The size of the light transmitter 71 is substantially the same as that of the hollow portion 83. A light diffuser 73 is disposed substantially at the center of the light transmitter 71.

In this modification, the hollow portion 83 can be easily processed. In this modification, when the light transmitter 71 is incorporated in the hollow portion 83, the light diffuser 73 can be easily and reliably placed on the central axis C.

[Modification 2]

As shown in FIG. 5B, a light transmitter 71 has a substantially spherical shape and engages with a hollow portion 83 having a truncated conical shape. The light transmitter 71 may have a substantially columnar shape. In the hollow portion 83, a space between the exit end face 50c of a light guide 50 and a first light converter 70 may be filled with air, or a light transmitter 75 may be disposed therein. The light transmitter 75 includes a material through which the primary light PL and the secondary light SL are transmitted. The light transmitter 75 is, for example, of a transparent silicone resin or a transparent epoxy resin. The light transmitter 75 may be disposed between the first light converter 70 and a holder exit portion 83b.

In this modification, light transmitters 71 having various shapes may be combined with the hollow portion 83 having a truncated conical shape. The hollow portion 83 may have an oval shape.

[Modifications 3 and 4]

For Modification 3, as shown in FIG. 5C, on the central axis C, the distance between a light diffuser 73 and a holder entrance portion 83a may be the same as the distance between the light diffuser 73 and a holder exit portion 83b. That is, the light diffuser 73 may be disposed at the middle of the holder exit portion 83b and the holder entrance portion 83a on the central axis C. Alternatively, for Modification 4, as shown in FIG. 5D, on the central axis c, the distance between the light diffuser 73 and the holder entrance portion 83a may be longer than the distance between the light diffuser 73 and the holder exit portion 83b. That is, the light diffuser 73 may be disposed between the holder exit portion 83b and the holder entrance portion 83a so as to be nearer the holder exit portion 83b than the holder entrance portion 83a. In this case, the density of the light diffuser 73 is preferably high. The density indicates the degree of diffusion. When the density is high, the effect of widening the light distribution is large. When the density is low, the effect of widening the light distribution is small. With this, as shown in FIG. 5D, the secondary light SL is also emitted to the rear (behind) of the light diffuser 73. In this modification, in the direction orthogonal to the central axis C of the primary light PL, the area of the light diffuser 73 is larger than the area of the light diffuser 73 of the first embodiment according to the spread of the primary light PL. For example, the diameter of the light diffuser 73 is larger than the diameter of the core 50d of the light guide 50.

In this modification, desired light-distribution characteristics of illumination light IL can be obtained depending on the position of the light diffuser 73. For example, with the configuration shown in FIG. 5D, the light-distribution angle of the illumination light IL can be wider than that in the first embodiment. In the configuration shown in FIG. 5D, primary light PL can be converted, by the light diffuser 73, into secondary light SL with a light-distribution angle being sufficiently widened and involving sufficiently reduced speckle, and part of the secondary light SL can be reflected by a reflecting member 85. Therefore, this allows the secondary light SL emitted from the light diffuser 73 to have a narrow angle, and the illuminating light IL with sufficiently reduced speckles to be emitted.

In this modification, the reflecting member 85 reflects at least part of the secondary light SL toward the front of the reflecting member 85, and converts a radiation angle of the secondary light SL to be narrower than the radiation angle of the original secondary light SL.

The light diffuser 73 is formed within the light transmitter 71. Therefore, part of the secondary light SL travels toward the rear of the light diffuser 73 and toward the holder entrance portion 83a (entrance face 71a) side of the reflecting member 85. After the secondary light SL is reflected by the reflecting member 85, the secondary light SL travels toward the holder exit portion 83b (exit face 71b) without entering the light diffuser 73 again by the reflection. Then, the secondary light SL is emitted as illumination light IL from the holder exit portion 83b (exit face 71b). In this way, the secondary light SL can be efficiently emitted as illumination light IL.

[Modifications 5 and 6]

As shown in FIGS. 5E and 5F, in this modification, it is assumed that three light diffusers 731, 733, and 735 are arranged. The light diffusers 731, 733, and 735 are arranged apart from each other in a central axis C direction.

As shown in FIG. 5E, for Modification 5, for example, the quantity of light diffusion in a first light diffuser 731 disposed near the holder entrance portion 83a is smaller than the quantity of light diffusion in a second light diffuser disposed away from the holder entrance portion 83a. The quantity of light diffusion in the second light diffuser 733 is smaller than the quantity of light diffusion in a third light diffuser 735 disposed near the holder exit portion 83b. Therefore, for example, in the direction orthogonal to the central axis C of the primary light PL, the area of the second light diffuser 733 is larger than that of the first light diffuser 731, and the area of the third light diffuser 735 is larger than that of the second light diffuser 733.

As shown in FIG. 5E, as Modification 6, the first light diffuser 731 diffuses part of illuminated primary light PL to generate a first secondary light SL1, which is diffused light. The part of the secondary light SL1 radiates the second light diffuser 733, so as to be diffused by the second light diffuser 733. Then, second secondary light SL2 is generated. The quantity of light diffusion of the second secondary light SL2 is larger than the quantity of light diffusion of the first secondary light SL1. Part of the secondary light SL2 radiates a third light diffuser 735, so as to be diffused by the third light diffuser 735. Then, third secondary light SL3 is generated. The quantity of light diffusion of the third secondary light SL3 is larger than the quantity of light diffusion of the second secondary light SL2. The secondary light SL3 is emitted as illumination light IL to the outside from the holder exit portion 83b.

The primary light PL that has not been diffused by the first light diffuser 731 may radiate a second light diffuser 733, so as to be converted into the second secondary light SL2 by the second light diffuser 733, which is not illustrated, though. The primary light PL and the first secondary light SL1 that have not been diffused by the second light diffuser 733 may radiate the third light diffuser 735, so as to be converted into the third secondary light SL3 by the third light diffuser 735. The primary light PL, the first secondary light SL1, and the second secondary light SL2 that have not been diffused by the third light diffuser 735 may be emitted as illumination light IL to the outside from the holder exit portion 83b.

As shown in FIG. 5F, the light diffusers 731, 733, and 735 may have an area identical to each other. The light diffusers 731, 733, and 735 have a density different from each other. For example, the density of the first light diffuser 731 is lower than that of the second light diffuser 733, and the density of the second light diffuser 733 is lower than that of the third light diffuser 735. The density is adjusted by, for example, the sizes of the light diffusers 731, 733, and 735, the shapes of the light diffusers 731, 733, and 735, the condensed spot diameter, the energy density, and the irradiation time of the laser light used for laser processing. By virtue of a difference in density, the light-distribution angle of the illumination light IL can be adjusted, and the intensity distribution of the illumination light IL can be adjusted.

In this modification, the number of diffusion times can be adjusted and the light distribution of the illumination light IL can be adjusted according to the number of light diffusers 73. The diffusion is conducted multiple times by the light diffusers 731, 733, and 735. Therefore, it is possible to provide illumination light IL having a wider light-distribution angle and to adjust the intensity distribution of the illumination light IL in the holder emission section 83b.

### Second Embodiment

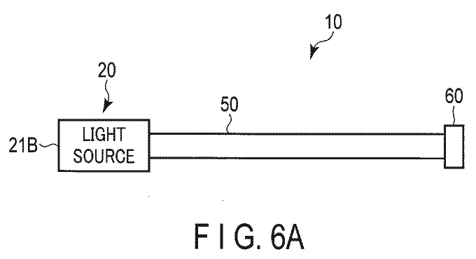

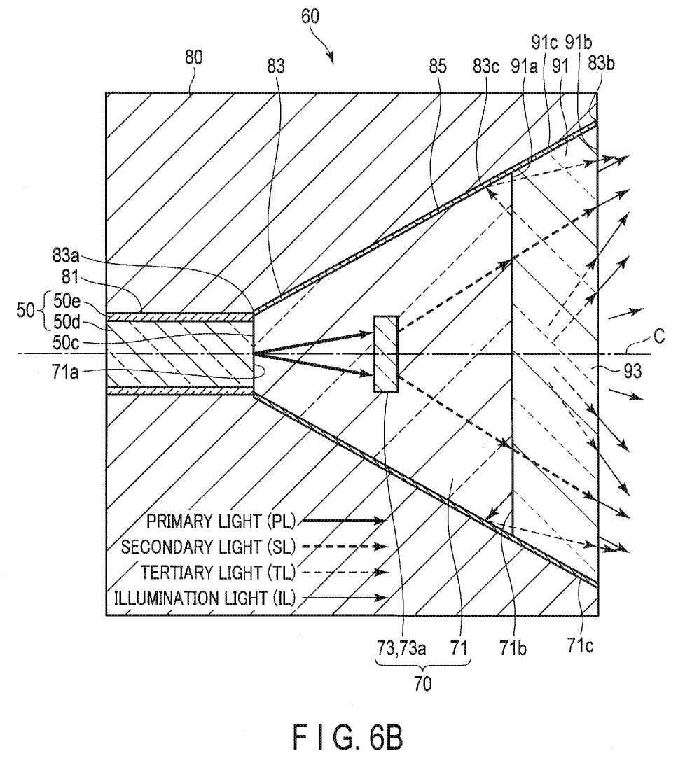

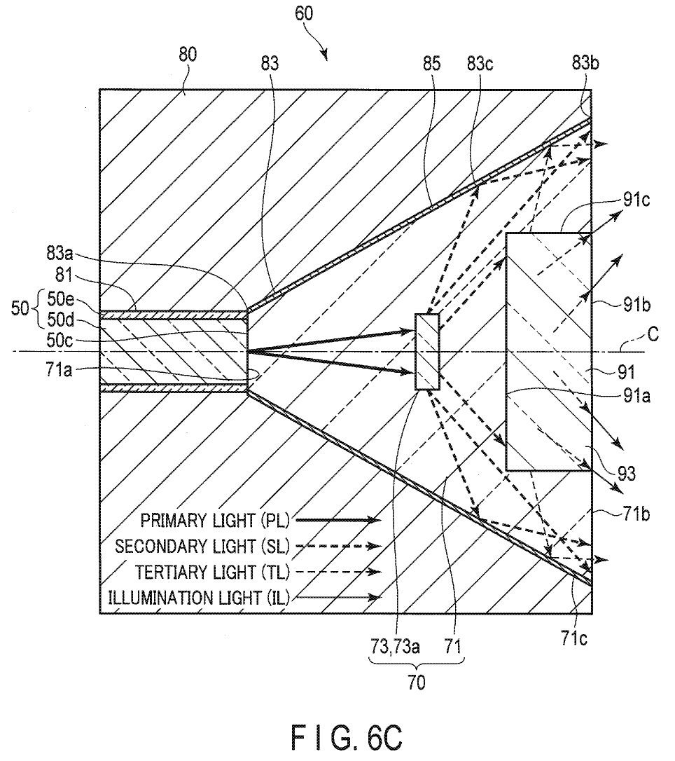

Hereinafter, a second embodiment of the present invention will be described with reference to FIGS. 6A and 6B. In the present embodiment, only differences from the first embodiment will be described.

As shown in FIG. 6A, a light source unit 20 emits, as primary light PL, a beam of laser light having one wavelength. For example, the light source unit 20 includes a light source 21B. The light source 21B is directly optically coupled to a light guide 50.

As shown in FIG. 6B, an illuminating unit 60 includes, in the central axis C direction, a second light converter 91 disposed between a light diffuser 73 and a holder exit portion 83b. Specifically, for example, the second light converter 91 is disposed between an exit face 71b of a first light converter 70 and a holder exit portion 83b. The second light converter 91 is disposed within a holder 80 and is held by the holder 80.

When receiving primary light PL or secondary light SL, the second light converter 91 converts the optical characteristics of at least part of received light to generate tertiary light TL to be included in illumination light IL. The second light converter 91 emits the generated tertiary light TL as part of the illumination light IL. Part of the primary light PL and part of the secondary light SL may be transmitted through the second light converter 91 without being converted into the tertiary light TL to be emitted as part of illumination light IL. Therefore, the illumination light IL of the present embodiment includes the primary light PL, the secondary light SL, and the tertiary light TL.

The second light converter 91 includes a light-distribution angle-converting member configured to generate tertiary light TL having a light-distribution angle wider than that of the received light (e.g., secondary light). For example, the second light converter 91 includes a wavelength-converting member 93 configured to generate tertiary light TL having a wavelength range different from the wavelength range of the received light (e.g., secondary light). The wavelength-converting member 93 includes, for example, a phosphor. The phosphor is, for example, a yellow phosphor configured to be excited by blue laser light (primary light PL or secondary light SL) of 445 nm, emitting yellow fluorescence (tertiary light TL). Since the generated fluorescence also travels in directions other than the forward direction, the phosphor can also be said to be a diffusing member in a broad sense. In the present embodiment, part of the primary light PL and part of the secondary light SL may be transmitted through the second light converter 91 without the wavelength thereof being converted by the second light converter 91. Therefore, the holder exit portion 83b (exit face 71b) may emit white illumination light IL in which yellow fluorescence and diffused blue laser light are mixed.

When the light diffuser 73 of the first light converter 70 generates the secondary light SL from the primary light PL, and when the light diffuser 73 generates the tertiary light TL from the light (e.g., secondary light) received by the second light converter 91, the calorific value of the second light converter 91 accompanied by the generation is larger than the calorific value of the light diffuser 73 of the first light converter 70 accompanied by the generation.

The second light converter 91 has a truncated conical shape, and the entirety of an outer circumferential surface 91c of the second light converter 91 is connected to the reflecting member 85. The exit face 71b of the first light converter 70 is stacked on an entrance face 91a of the second light converter 91, and an exit face 91b of the second light converter 91 is disposed at the holder exit portion 83b. The entrance face 91a is smaller than the exit face 91b. The entrance face 91a is larger than the area of the light diffuser 73 in a direction orthogonal to the central axis C. The entrance face 91a is disposed away from the light diffuser 73 and is disposed between the light diffuser 73 and the holder exit portion 83b. The entrance face 91a and the exit face 91b are, for example, circular in shape. For example, the primary light PL and the secondary light SL enter the entrance face 91a, and the illumination light IL is emitted to the exit surface 91b. The second light converter 91 is thermally connected to the first light converter 70 and the holder 80.

[Operation]

The primary light PL emitted from the light source 21B is guided to the illuminating unit 60 by the light guide 50.

The primary light PL is emitted from the exit end face 50c toward the first light converter 70.

The primary light PL enters the light transmitter 71 from the holder entrance portion 83a (entrance face 71a) and travels within the light transmitter 71. The primary light PL travels toward the light diffuser 73. After part of the primary light PL is transmitted through the light transmitter 71, transmitted through the light diffuser 73, travels toward the reflecting member 85 without being diffused by the light diffuser 73, and is reflected by the reflecting member 85, the part of the primary light PL may travel toward the second light converter 91 without entering the light diffuser 73, which is not illustrated, though. Part of the primary light PL may be transmitted through the light transmitter 71, transmitted through the light diffuser 73, and travel directly toward the second light converter 91 without being diffused by the light diffuser 73, which is not illustrated, though.

At least part of the primary light PL that has entered the light diffuser 73 is converted into secondary light SL having a different light-distribution angle from that of the primary light PL, without the wavelength of the primary light PL being changed by the light diffuser 73. At least part of the secondary light SL travels within the light transmitter 71 and travels directly toward the second light converter 91. Part of the secondary light SL may travel toward the second light converter 91 after being reflected by the reflecting member 85, which is not illustrated, though.

The second light converter 91 is irradiated with part of the primary light PL, not shown, and the secondary light SL to convert the optical characteristics of at least part of the irradiated light, generating tertiary light TL. Part of the primary light PL and part of the secondary light SL may be transmitted through the second light converter 91 without the wavelength thereof being converted by the second light converter 91. The primary light PL, the secondary light SL, and the tertiary light TL are emitted as illumination light IL from the holder exit portion 83b (exit face 91b) toward the outside. The illumination light IL is emitted forward from the holder exit portion 83b (exit face 91b).

Part of the tertiary light TL travels from the second light converter 91 toward the rear of the second light converter 91 and toward the holder entrance portion 83a (entrance face 71a) side of the reflecting member 85. The holder entrance portion 83a (entrance face 71a) side of the reflecting member 85 indicates a portion of the reflecting member 85 disposed between the second light converter 91 and the holder entrance portion 83a in the central axis C direction. The tertiary light TL travels toward the second light converter 91 after being reflected by this portion of the reflecting member 85. Then, the tertiary light TL is transmitted through the second light converter 91, and then emitted, as illumination light IL, forward from the holder exit portion 83b (exit face 91b).

[Effect]

In the present embodiment, since the second light converter 91 is used, it is possible to reduce the number of light sources and to reduce the cost of the illuminating device 10. In the present embodiment, since the second light converter 91 is disposed within the holder 80, the second light converter 91 can be protected. Therefore, it is possible to provide the illuminating device 10 with fewer fluctuations in light-distribution characteristics with respect to externally applied loads including an external force, thereby providing the illuminating device 10 with a high degree of reliability.

When the second light converter 91 including a phosphor converts the wavelength, since the light radiating the second light converter 91 changes into heat at a constant ratio, the second light converter 91 generates heat. Particularly when the second light converter 91 is directly irradiated with the primary light PL, which is laser light having a high-energy density, the irradiation area in the second light converter 91 to be irradiated with the laser light generates heat. That is, the second light converter 91 generates heat locally. Then, the second light converter 91 itself or a circumferential member (e.g., light transmitter 71) of the second light converter 91 is burned by heat. As a result, the second light converter 91 and the circumferential member may cause a problem by being broken due to the burning in some cases. In order to avoid this problem, it is necessary to widen an irradiation area in the second light converter 91 to be irradiated with the laser light, and to irradiate the second light converter 91 with light having a low-energy density.

Here, it is assumed that the light diffuser 73 is not provided and the illuminating unit 60 is sufficiently large. In this case, even if the light-distribution angle of the primary light PL is narrow, the distance between the exit end face 50c and the second light converter 91 is elongated. Therefore, the irradiation area in the second light converter 91 to be irradiated with the primary light PL is widened, and the second light converter 91 will be irradiated with light having a low-energy density. Therefore, the above-mentioned problem is avoided. However, when the illuminating unit 60 is disposed in a narrow space within the distal end section of an insertion section 120, for example, it is necessary to reduce the size of the illuminating unit 60. Therefore, the distance between the exit end face 50c and the second light converter 91 is compelled to shorten, the irradiation area in the second light converter 91 to be irradiated with the primary light PL is narrowed, and the second light converter 91 is compelled to be irradiated with light having a high-energy density. In this instance, the above-mentioned problem occurs.

Therefore, in the present embodiment, at least part of the light diffuser 73 is placed between the exit end face 50c and the second light converter 91 in the central axis C direction, and the light diffuser 73 diffuses at least part of the primary light PL. Therefore, even if the distance between the exit end face 50c and the second light converter 91 is short, it is possible to widen an irradiation area in the second light converter 91 to be irradiated with the primary light PL, as compared with a state where the second light converter 91 is directly irradiated with the primary light PL, and to irradiate the second light converter 91 with light having a low-energy density as compared with the primary light PL. In this instance, the above-mentioned problem can be avoided, laser light with high power output can be used, and illumination light IL with high power output can be obtained.

The calorific value of the second light converter 91 is larger than the calorific value of the first light converter 70. Therefore, heat generated from the second light converter 91 can be transmitted to the light transmitter 71, and damage to the second light converter 91 due to the heat can be prevented. Furthermore, the second light converter 91 is directly thermally connected to the holder 80 and is thermally connected to the holder 80 through the light transmitter 71. Therefore, heat generated from the second light converter 91 can be transmitted to the holder 80, and damage to the second light converter 91 due to the heat can be prevented.

[Modification 1]

As shown in FIG. 6C, the second light converter 91 may have a columnar shape. The outer circumferential face 91c of the second light converter 91 is surrounded by the light transmitter 71, is in contact with the light transmitter 71, and is disposed away from the inner circumferential face 83c of the holder 80. Therefore, the light transmitter 71 is lateral to the second light converter 91.

Part of the secondary light SL may be transmitted through the light transmitter 71 and travel directly toward the holder exit portion 83b through the light transmitter 71, which is lateral to the second light converter 91, without entering the second light converter 91. Part of the secondary light SL may be reflected by the reflecting member 85 and then travel toward the holder exit portion 83b through the light transmitter 71, which is lateral to the second light converter 91, without entering the second light converter 91. Then, the secondary light SL may be emitted as illumination light IL.

Part of tertiary light TL may be reflected by the reflecting member 85 through the light transmitter 71, which is lateral to the second light converter 91, and travel directly toward the holder exit portion 83b. Then, the tertiary light TL may be emitted as illumination light IL. In this modification, the tertiary light TL can be extracted efficiently.

### Third Embodiment

Hereinafter, a third embodiment of the present invention will be described. In the present embodiment, only differences from the first and second embodiments will be described.

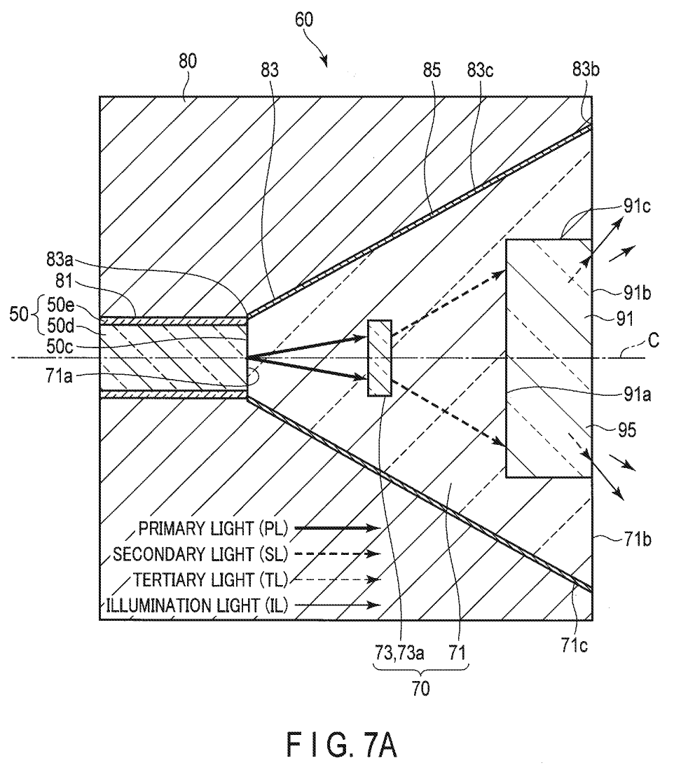

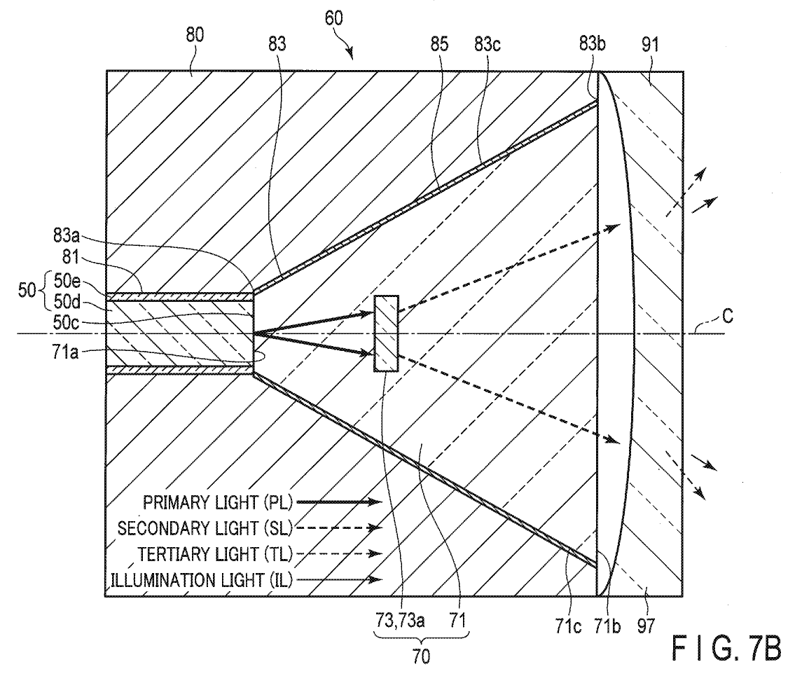

As shown in FIG. 7A, a second light converter 91 includes a diffusing member 95 configured to diffuse received light (e.g., secondary light) to generate tertiary light TL. The received light indicates part of the primary light PL and the secondary light SL. The diffusing member 95 includes diffusing particles (not shown) and a containing material (not shown) containing the diffusing particles.

The diffusing particles are dispersed in the containing material and sealed by the containing material. The diffusing particles are fine particles formed, for example, with a metal or a metal compound. Such diffusing particles are, for example, alumina, titanium oxide, barium sulfate, etc. The particle size of the diffusing particles ranges from several hundred nanometers (nm) to tens of micrometers (μm). The refractive index of the diffusing particles is different from the refractive index of the containing material. For example, the refractive index of the diffusing particles is preferably higher than that of the containing material. With this configuration, the diffusing member can improve the diffusivity of light.

The containing material is formed with a material through which the primary light PL and the secondary light SL are transmitted. The containing material is, for example, a transparent glass, a transparent silicone-based resin, or a transparent epoxy-based resin. The containing material has a high transmittance with respect to the primary light PL and the secondary light SL. The containing material seals members included in the containing material.

The light-distribution angle of the diffusing member 95 is controlled, for example, by the concentration of the diffusing particles relative to the containing material, the thickness of the diffusing member, etc.

In the present embodiment, the light-distribution angle of the received light can be further widened by the diffusing member 95.

[Modification 1]

As shown in FIG. 7B, the second light converter 91 is disposed in front of the holder exit portion 83b in the central axis C direction. The second light converter 91 may include a lens 97 configured to diffuse received light (e.g., secondary light), generating tertiary light TL. The second light converter 91 is detachable from the holder exit portion 83b.

The second light converter 91 is a concave lens that is concaved from the holder exit portion 83b toward the outside. The concave lens further widens the light distribution of the secondary light SL. The second light converter 91 may have a convex lens that is convexed from the holder exit portion 83b toward the outside and configured to narrow the light distribution of the secondary light SL.

The present invention is not limited to the above-mentioned embodiments themselves, and the structural elements can be modified and embodied in the implementation stage without departing from the spirit and scope thereof. Furthermore, various inventions can be formed by appropriately combining the plurality of structural elements disclosed in the above-mentioned embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An illuminating device comprising:
a light source unit configured to emit primary light; and
an illuminating unit configured to generate illumination light based on the primary light, emitting the illumination light to an opposite side of the light source unit,
the illuminating unit comprising:
a first light converter configured to convert optical characteristics of at least part of the primary light; and
a holder internally holding the first light converter,
the holder having:
a holder entrance portion where the primary light enters; and
a holder exit portion where the illumination light exits,
the first light converter comprising:
a light transmitter through which the primary light is transmitted; and
at least one light diffuser formed within the light transmitter, the light diffuser being configured to diffuse the at least part of the primary light traveling within the light transmitter, generating secondary light included in the illumination light,
the light diffuser having a refractive index different from a refractive index of the light transmitter, and
the light diffuser having any one of a hole formed within the light transmitter, a refractive index-modifying portion, and a crack portion.

2. The illuminating device according to claim 1, wherein the light diffuser has a substantially columnar shape or a substantially spherical shape.

3. The illuminating device according to claim 1, further comprising a light guide configured to guide the primary light emitted from the light source unit to the illuminating unit,
wherein the light diffuser is configured to generate the secondary light having a light-distribution angle equal to or larger than an NA of the light guide.

4. The illuminating device according to claim 3, wherein the holder has a hollow portion that communicates with the holder entrance portion and the holder exit portion and in which the light transmitter is disposed, and
the hollow portion has a truncated conical shape gradually expanding from the holder entrance portion toward the holder exit portion, and the light transmitter has a truncated conical shape or a substantially spherical shape, or the hollow portion has a substantially columnar shape, and the light transmitter has a substantially columnar shape, and
at least part of the light diffuser is formed on a central axis of the primary light that enters the light transmitter from the holder entrance portion.

5. The illuminating device according to claim 4, wherein the light diffuser is disposed nearer the holder entrance portion than the holder exit portion, between the holder exit portion and the holder entrance portion, and
the light diffuser has a diameter identical to or smaller than a diameter of the light guide.

6. The illuminating device according to claim 4, wherein the light diffuser is disposed on a plane orthogonal to the central axis, and a length of the light diffuser on the central axis is shorter than a length of the light diffuser in a direction orthogonal to the central axis.

7. The illuminating device according to claim 4, wherein the light diffuser is disposed substantially at a center of the light transmitter.

8. The illuminating device according to claim 4, wherein the light diffuser comprises a first light diffuser and a second light diffuser,
the first and second light diffusers are arranged away from each other along the central axis, and
a quantity of light diffusion in the first light diffuser disposed near the holder entrance portion is smaller than a quantity of light diffusion in the second light diffuser disposed away from the holder entrance portion.

9. The illuminating device according to claim 8, wherein in a direction orthogonal to the central axis, an area of the second light diffuser is larger than an area of the first light diffuser.

10. The illuminating device according to claim 8, wherein the first light diffuser has a density lower than that of the second light diffuser.

11. The illuminating device according to claim 1, wherein the holder has a hollow portion that communicates with the holder entrance portion and the holder exit portion and in which the light transmitter is disposed,
the hollow portion has a truncated conical shape gradually expanding from the holder entrance portion toward the holder exit portion, and
the holder includes a reflecting member that is disposed on a tapered inner circumferential face of the holder, and configured to reflect the primary light and the secondary light toward the holder exit portion.

12. The illuminating device according to claim 11, wherein the reflecting member is configured to reflect at least part of the secondary light, converting a radiation angle of the secondary light into a narrow angle.

13. The illuminating device according to claim 11, wherein the reflecting member is configured to reflect part of the secondary light so that the part of the secondary light traveling from the light diffuser toward the holder entrance portion travels to the holder exit portion without entering the light diffuser again.

14. The illuminating device according to claim 1, wherein the illuminating unit comprises a second light converter configured, when receiving the primary light or the secondary light, to convert optical characteristics of at least part of the received light to generate tertiary light to be included in the illumination light, and, in a central axis direction of the primary light entering the light transmitter from the holder entrance portion, the illuminating unit is disposed between the light diffuser and the holder exit portion, or is disposed in front of the holder exit portion, the front indicating the opposite side of a position where the light source unit is disposed.

15. The illuminating device according to claim 14, wherein when the first light converter generates the secondary light from the primary light, and when the second light converter generates the tertiary light from the secondary light, a calorific value of the second light converter accompanied by the generation of the tertiary light is greater than a calorific value of the first light converter accompanied by the generation of the second light.

16. The illuminating device according to claim 14, wherein the second light converter comprises a light-distribution angle-converting member configured to generate the tertiary light having a light-distribution angle wider than a light distribution of the secondary light.

17. The illuminating device according to claim 14, wherein the second light converter comprises a wavelength-converting member configured to generate the tertiary light having a wavelength range different from a wavelength range of the secondary light.

18. The illuminating device according to claim 14, wherein the second light converter comprises a diffusing member or a lens configured to diffuse the secondary light, generating the tertiary light.

19. The illuminating device according to claim 1, wherein the light transmitter comprises a glass or an acrylic.

20. The illuminating device according to claim 1, wherein the light source unit is configured to emit a beam of laser light having a wavelength or beams of laser light each having a wavelength, as the primary light, or emit beams of laser light having different wavelengths from each other, as the primary light.

21. The illuminating device according to claim 20, wherein the light source unit includes a light combiner configured to combine the beams of laser light having different wavelengths from each other.
